(19) United States
(12) Patent Application Publication
(10) Pub. No.: US 2023/0330657 A1
(43) Pub. Date: Oct. 19, 2023

(54) SAMPLE PREPARATION AND DETECTION SYSTEMS AND METHODS

(71) Applicant: Charm Sciences, Inc., Lawrence, MA (US)

(72) Inventors: Robert S Salter, Reading, MA (US); Richard Skiffington, North Reading, MA (US); Shishan Wang, Andover, MA (US)

(73) Assignee: Charm Sciences, Inc., Lawrence, MA (US)

(21) Appl. No.: 18/027,096

(22) PCT Filed: Sep. 27, 2021
(86) PCT No.: PCT/US2021/052157

§ 371 Date: Mar. 18, 2023

Related U.S. Application Data
(60) Provisional application No. 63/193,198, filed on May 26, 2021.
(60) Provisional application No. 63/123,689, filed on Dec. 10, 2020.
(60) Provisional application No. 63/084,712, filed on Sep. 29, 2020.
(60) Provisional application No. 63/083,468, filed on Sep. 25, 2020.

(51) Int. Cl. B01L 3/00; C12Q 1/6844; C12Q 1/689

(52) U.S. Cl. CPC B01L 3/5023; C12Q 1/6844; C12Q 1/689; B01L 2200/0689; B01L 2200/16; B01L 2300/069; B01L 2300/0825

(57) ABSTRACT

Sample preparation and detection devices, methods, and assemblies are shown and described. In one embodiment, an assembly for preparing a sample for predicting a bacterial presence includes a compartmentalized container having a sample collector reservoir, a first storage compartment, and a second storage compartment. The result is improved efficient and effective predicting of a bacterial presence.

This application claims the benefit of PCT/US2152157, filed Sep. 27, 2021; U.S. Provisional Application No. 63/083468, filed Sep. 25, 2020; U.S. Provisional Application No. 63/084712, filed Sep. 29, 2020; U.S. Provisional Application No. 63/123689, filed Dec. 10, 2020; and U.S. Provisional Application No. 63/193198, filed May 26, 2021, all of which are incorporated herein by reference in their entireties.

### FIELD OF THE TECHNOLOGY

The present disclosure relates generally to the detection of bacteria, and more particularly to improved test devices, methods, and assemblies for the preparation of a sample and analysis of a sample.

### SUMMARY

In accordance with the present disclosure, sample preparation and detection systems and assemblies are provided for a wide variety of applications. This disclosure provides improved systems and methods that are convenient, efficient, and safe, for instance in preparing samples for predicting a bacterial presence, when present, in a sample.

In one embodiment, an assembly for detecting bacteria, when present, in a sample, comprises a sample collector compartment cooperatively-engaged with a proximate opening and adapted to receive a sample; a media compartment cooperatively-engaged with the sample collector compartment and housing a media in a storage position, wherein the sample collector compartment adapted to releasably introduce the sample into the media compartment to define a first admixture; an amplification compartment cooperatively-engaged with the media compartment, wherein the media compartment adapted to releasably introduce the first admixture into the amplification compartment and support phage amplification to define a luminescent enzyme; and a disinfectant compartment cooperatively-engaged with the amplification compartment and adapted to releasably introduce a disinfectant.

In certain examples, the phage amplification may be adapted to target at least one specific bacterial receptor, insert into an enzyme operon, replicate at least one specific nucleic acid sequence. In certain examples, the assembly includes concentration of the phages, for instance a concentrator to concentrate the phages. In certain examples, the assembly may include a centrifugation device, or the like, to concentrate via centrifugation or similar concentration to enhance any of the enzyme detection shown and described herein. In certain examples, the assemble includes a luciferase assay substrate to assist sample preparation to detect any of the luminescent signals shown and described herein.

In one embodiment, a compartmentalized assay development device comprises a sample collector reservoir cooperatively-engaged with an opening and adapted to receive a sample; a media rehydrating compartment cooperatively-engaged in communication capability with the sample collector reservoir and housing a media; and a phage amplification compartment cooperatively-engaged in communication capability with the media rehydrating compartment, and wherein the compartmentalized assay development device adapted to expose a phage amplification enclosure and prepare the sample for detection of bacteria, when present, in the sample.

In certain examples, the sample collector reservoir houses assay components in a storage position, for instance to releasably introduce the assay components in an admixture; the media rehydrating compartment houses assay components in a storage position; and the phage amplification compartment houses assay components in a storage position, for instance to releasably introduce the assay components in an admixture. The sample collector reservoir may house assay components in a storage position and adapted to releasably introduce the assay components in an admixture; and The phage amplification compartment may house recombinant phage assay components in a storage position and adapted to releasably introduce the assay components in an admixture.

In particular examples, the device may include at least one, including a first, temporary barrier between the sample collector reservoir and the media rehydrating compartment. The device may include at least one, including a second, temporary barrier between the media rehydrating compartment and the phage amplification compartment. The device may include at least one, including a third, temporary barrier between a fourth compartment and a third compartment.

In certain examples, the device may include a phage amplification enclosure that is housed in the media rehydrating compartment. In the alternative, or in addition to, the phage amplification enclosure may be housed in the phage amplification compartment. Further, those skilled in the art having the benefit of this disclosure will recognize additional compartments, locations, or arrangements for housing the phage amplification enclosure.

In particular examples, the proximate opening may include a perforated seal. The recloseable proximate opening may include a closure assembly. The closure assembly may include a fastener. The fastener may include at least one twister. The closure assembly may include a leak-proof seal.

In certain examples, the assay components may include a disinfectant. The disinfectant may be a virus, bacteria, or the like disinfectant. For instance, the disinfectant neutralizer may include about ten milligrams of sodium thiosulfate.

In particular examples, the prepared sample for detection of bacteria may be adapted for detection of a recombinant luciferase enzyme. The recombinant luciferase enzyme may be an insert into an infecting phage and being replicated as part of a virus replication. The phage amplification enclosure may target at least one specific bacterial receptor, insert into an enzyme operon, replicate at least one specific nucleic acid sequence.

In particular examples, the phage amplification enclosure includes a nanoluciferase. For instance, the nanoluciferase comprises an indicator gene and a bacteriophage late promoter adapted to control transcription of the indicator gene.

In one embodiment, an assembly for detecting bacteria in a sample comprises a compartmentalized container having a sample collector reservoir; a first storage compartment cooperatively-engaged with the sample collector reservoir, and housing assay components in a storage position and releasably introducing the assay components into the sample collector reservoir to define a first admixture; an second compartment cooperatively-engaged with the first storage compartment, wherein the first storage compartment adapted to releasably introduce the first admixture into the second compartment and support a phage amplification to define specific nucleic acid sequences; and a third storage compartment in operative communication with the second storage compartment, and housing ribonucleic acid (RNA) or deoxyribonucleic acid (DNA) extraction reagents in a storage position and releasably introducing the extraction reagents into the first admixture to define a third admixture, and wherein the assembly adapted to prepare the sample for rapidly predicting a bacterial presence, when present, in the sample.

In particular examples, the assay components include a bactericide. The phage amplification enclosure may target a bacterial enzyme operon, or a specific viral nucleic acid sequence. The phage amplification enclosure may include a nanoluciferase. The nanoluciferase may include an indicator gene and a bacteriophage late promoter adapted to control transcription of the indicator gene. In certain examples, the assemble includes a luciferase assay substrate to assist sample preparation to detect any of the luminescent signals shown and described herein.

In one embodiment, an assembly for detecting bacteria, when present, in a sample, includes a sample collector compartment cooperatively-engaged with a proximate opening and adapted to receive a sample; a media compartment cooperatively-engaged with the sample collector compartment and housing a media in a storage position, wherein the sample collector compartment adapted to releasably introduce the sample into the media compartment to define a first admixture; an amplification compartment cooperatively-engaged with the media compartment, wherein the media compartment adapted to releasably introduce the first admixture into the amplification compartment and support phage amplification to define a luminescent substrate; and a bactericide compartment cooperatively-engaged with the amplification compartment and adapted to releasably introduce a bactericide.

In particular examples, the phage amplification adapted to target a bacterial enzyme operon, or a specific viral nucleic acid sequence.

In one embodiment, an assembly for detecting bacteria in a sample includes a compartmentalized container having a sample collector reservoir; a first storage compartment cooperatively-engaged with the sample collector reservoir, and housing assay components in a storage position and releasably introducing the assay components into the sample collector reservoir to define a first admixture; a second storage compartment in operative communication with the sample collector reservoir, and housing ribonucleic acid (RNA) or deoxyribonucleic acid (DNA) extraction reagents in a storage position and releasably introducing the extraction reagents into the first admixture to define a second admixture, and wherein the assembly adapted to prepare the sample for rapidly predicting a bacterial presence, when present, in the sample.

In certain examples, the assay components include a culture media. The bacterial presence may include a coliform in a dairy product selected from the group consisting of a raw milk and a pasteurized dairy product. The bacterial presence may include E.coli in a sample selected from the group consisting of a food product and a water. The assembly may be adapted to target adenosine triphosphate (ATP) kinase.

In certain examples, the assembly may include a clustered regularly interspaced short palindromic repeat (CRISPR) treatment system adapted to initiate a CRISPR of the RNA or DNA bound loop complexes.

In one embodiment, an assembly for detecting bacteria in a sample includes a compartmentalized container having a sample collector reservoir; a first storage compartment cooperatively-engaged with the reservoir, and housing assay components in a storage position and releasably introducing the assay components into the reservoir to define a first admixture; a second storage compartment in operative communication with the reservoir, and housing ribonucleic acid (RNA) or deoxyribonucleic acid (DNA) extraction reagents in a storage position and releasably introducing the extraction reagents; a second container adapted to receive a portion of the admixture and having: a bacteriophage adapted to target a bacterial enzyme operon, or a specific viral nucleic acid sequence, thereby logarithmically multiplying at least one target RNA or DNA, and wherein the assembly adapted to prepare the sample for rapidly predicting a bacterial presence, when present, in the sample.

In certain examples, the assay components may include a culture media. The assay components include a culture broth. The assay components may include a phage. The sample collector reservoir may include a filter. The assembly may include a multicompartment niblet, for instance having a moisture impervious layer face and a permeable membrane face.

In certain examples, the bacterial presence may include a coliform in a dairy product selected from the group consisting of a raw milk and a pasteurized dairy product. The bacterial presence may include E.coli in a sample selected from the group consisting of a food product and a water. The assembly may target adenosine triphosphate (ATP) kinase.

In one embodiment, a compartmentalized assay development device includes a recloseable proximate opening; a sample collector reservoir cooperatively-engaged with the proximate opening and adapted to receive a sample; a media rehydrating compartment cooperatively-engaged in a fluid communication capability with the sample collector reservoir and housing a media; and a phage amplification compartment cooperatively-engaged in a fluid communication capability with the media rehydrating compartment and adapted to expose a phage amplification enclosure, and wherein the compartmentalized assay development device adapted to prepare the sample for detection of bacteria, when present, in the sample.

In certain examples, the sample collector reservoir may house assay components in a storage position and may releasably introduce the assay components in an admixture; the media rehydrating compartment may house assay components in a storage position; and the phage amplification compartment may house assay components in a storage position and may releasably introduce the assay components in an admixture.

In certain examples, the device may include a first temporary barrier between the sample collector reservoir and the media rehydrating compartment. The device may include a second temporary barrier between the media rehydrating compartment and the phage amplification compartment. The device may include a phage amplification enclosure temporarily housed in the media rehydrating compartment.

In one embodiment, an assembly for detecting bacteria, when present, in a sample, comprises a recloseable proximate opening; a sample collector compartment cooperatively-engaged with the proximate opening and adapted to receive a sample; a media compartment cooperatively-engaged with the sample collector compartment and housing a media in a storage position, wherein the sample collector compartment adapted to releasably introduce the sample into the media compartment to define a first admixture; and an amplification compartment cooperatively-engaged with the media compartment, wherein the media compartment adapted to releasably introduce the first admixture into the amplification compartment and support phage amplification to define a luminescent substrate.

In one embodiment, an assembly for detecting bacteria, when present, in a sample, comprises a compartmentalized assay having: a sample collector compartment; a media compartment cooperatively-engaged with the sampling compartment and housing a media in a storage position, and adapted to combine the sample and media to define a first admixture; and an amplification compartment cooperatively-engaged with the media compartment, and adapted to releasably introduce the first admixture with a phage amplification enclosure to define a luminescent substrate; a vial adapted to receive at least a portion of the luminescent substrate; and a luminometer adapted to analyze the luminescent substrate in the vial for rapidly predicting a bacterial presence, when present, in the sample.

In one embodiment an assay device comprises a sampling compartment having a sealed opening; a media compartment adjacent the sampling compartment; a first temporary moisture impervious barrier between the sampling compartment and the media compartment; a phage amplification compartment adjacent the media compartment; and a second temporary moisture impervious barrier between the media compartment and the phage amplification compartment, and wherein the device adapted to prepare a phage-amplified luminescent substrate for a detection of an analyte, when present, in a sample.

In one embodiment a method for detecting bacteria, when present, in a sample, comprises collecting a sample in a first independent compartment in a compartmentalized container; introducing contents of the first independent compartment into an adjacent media compartment to define a first admixture; incubating the first admixture in the compartmentalized container; introducing the first admixture into an adjacent amplification compartment and exposing contents of a phage amplification enclosure to define a luminescent substrate; and collecting a portion of the luminescent substrate for detecting a luminescent signal.

In one embodiment a method for rapidly predicting a bacterial presence in a sample, comprises collecting a sample in a first independent compartment in a compartmentalized container; bursting the sample into an adjacent media compartment to define a first admixture; incubating the first admixture in the compartmentalized container; bursting the first admixture into an adjacent amplification compartment; releasing contents of a phage amplification enclosure to define a phage-amplified luminescent substrate; and collecting a portion of the luminescent substrate for detecting a luminescent signal.

In one embodiment a compartmentalized assay development device comprises a recloseable proximate opening; a sample collector reservoir cooperatively-engaged with the proximate opening and adapted to receive a sample; a media rehydrating compartment cooperatively-engaged in a fluid communication capability with the sample collector reservoir and housing a media; and a phage amplification compartment cooperatively-engaged in a fluid communication capability with the media rehydrating compartment and adapted to expose a phage amplification enclosure, and wherein the compartmentalized assay development device adapted to prepare the sample for detection of bacteria, when present, in the sample.

In certain examples, the device may include a first temporary barrier between the sample collector reservoir and the media rehydrating compartment. The first temporary barrier may include a moisture impervious barrier in a storage position. The first temporary barrier may include a burst liner. The device may include a second temporary barrier between the media rehydrating compartment and the phage amplification compartment. The second temporary barrier may include a moisture impervious barrier in a storage position. The second temporary barrier may include a burst liner.

In particular examples, the device may include a phage amplification enclosure temporarily housed in the media rehydrating compartment. Further, the device may include a phage amplification enclosure housed in the phage amplification compartment.

In certain examples, the proximate opening may include a perforated seal. The recloseable proximate opening may include a closure assembly. The closure assembly may include a fastener. The fastener may include at least one twister. The closure assembly may include a leak-proof seal.

In particular examples, the sample collector reservoir may house assay components in a storage position and adapted releasably introduce the assay components in an admixture. The assay components may include a disinfectant neutralizer. The disinfectant neutralizer may include about ten milligrams of sodium thiosulfate. The media rehydrating compartment may include assay components in a storage position. The media rehydrating compartment may include assay components in a storage position and adapted releasably introduce the assay components in an admixture. The phage amplification compartment may include assay components in a storage position and adapted releasably introduce the assay components in an admixture. The assay components may include a bactericide.

In certain examples, the phage amplification enclosure may target a bacterial enzyme operon, or a specific viral nucleic acid sequence. The phage amplification enclosure may include a puncturable packet. The phage amplification enclosure may include a puncturable vial. The phage amplification enclosure may include at least one buffer.

In particular examples, the device may include an enclosed distal portion. The device may produce a composition having luminescent signal, wherein a sample of the composition is aligned in a separate vial for analysis, including any luminesce chemistry and any luminometer. The luminescent signal may enable a semi-quantitative test result. The luminescent signal may be detectable by any handheld, benchtop, combination, or the like luminometer. The detection of bacteria may enable a rapid detection of generic E.coli. The detection of generic E.coli may be between about ten to about twenty five colony forming units per one hundred milliliters. The detection may include low level generic E.coli in about six to about seven hours.

In particular examples, the sample may be a potable drinking water, a waste water, an effluent, a surface water, a ground water, a swimming water, an irrigation water, a run off, an extracted soil, soil complement, and an extracted agricultural produce sample, the like, and a combination thereof.

In certain examples, the bacterial presence includes a shiga toxin producing E. coli selected from farm samples and extracts as well as the food groups consisting of a raw milk, ground meats, vegetable homogenates and washes. For instance, the shiga toxin producing E. coli may include O157H7 or the like. Those skilled in the art having the benefit of this disclosure will recognize additional enhanced detection levels supported by the embodiments and examples herein.

In particular examples, detection may include less than about ten CFU/100 mL coliform in the pasteurized dairy product. The device may include utilizing any luminescence chemistry. The device may include utilizing at least one T type bacteriophage, for instance a specific RNA or DNA sequence of the T type bacteriophage. Further, the sample collector reservoir may contain a filter.

In one alternative embodiment, an assembly for detecting bacteria in a sample includes a compartmentalized container having a sample collector reservoir; a first storage compartment cooperatively-engaged with the reservoir, and housing assay components in a storage position and releasably introducing the assay components into the reservoir to define a first admixture; a second storage compartment in operative communication with the reservoir and housing extraction reagents in a storage position and releasably introducing the extraction reagents into the first admixture to define a second admixture.

In certain alternative examples, the first storage compartment includes a moisture and vapor impervious layer enclosing the assay components. The second storage compartment may include a moisture and vapor impervious layer enclosing the extraction reagents. The second storage compartment may be cooperatively-engaged with the reservoir, a combination thereof, and the like. The assay components include any of the culture media, culture broth, induction agents, additives, reagents, and the like shown, described, and incorporated by reference herein. The assay components may include a magnetic bead. The magnetic bead may include a lectin, an antibody, a combination thereof and the like. Further, the assay components may include a phage.

In certain alternative examples, the bacterial presence includes E.coli in a sample selected from the group consisting of a food product and a water. The assembly may include a detection of greater than about one CFU/mL E.coli in about one hundred mL water, for instance a detection of E. coli in water within less than about six hours. The sample may include a potable drinking water, a waste water, an effluent, a surface water, a ground water, a swimming water, an irrigation water, a run off, a combination thereof, and the like. The assembly may include a detection greater than about one CFU/mL E. coli in about twenty-five grams of a food product. The assembly may include at least one T type bacteriophage. The assembly may include a specific RNA or DNA sequence of the T type bacteriophage. Those skilled in the art having the benefit of this disclosure will recognize additional enhanced detection levels supported by the embodiments and examples herein.

In one alternative embodiment, a method for rapidly predicting a bacterial presence in a sample includes collecting a sample in a compartmentalized container housing assay components in a first independent compartment, and extraction reagents in a second independent compartment; introducing the assay components from the independent compartment into a sample reservoir for infecting the sample.

In particular alternative examples, the method may include introducing the assay components into a sample reservoir via screwing, including, but not limited to, opposing threads of a cap about the container to break any seal, enclosure, or the like. The method may include extending a plunger, including two or more plungers, through any seal, enclosure, or the like to expose the first independent compartment. Further, introducing the assay components into a sample reservoir may include removing a seal on the first independent compartment. The seal may include a tab or the like. The method may include dissolving any of the assay components shown and described herein, including with agitation or without agitation.

In particular alternative examples, the method may include incubating the reservoir. The method may include incubating the reservoir between about thirty degrees Celsius to about fifty degrees Celsius. The method may include introducing a phage for targeting an area within the bacteria. The method may include removing a liquid concentrating the bacteria. The method may include introducing the extraction reagents via screwing opposing threads of a second independent cap about the container. The method may include introducing the extraction reagents via extending a plunger to perforate the second independent compartment. Further, the method may include introducing the extraction reagents into a sample reservoir via removing a seal on the second independent compartment. Those skilled in the art having the benefit of this disclosure will recognize additional enhanced detection levels supported by the embodiments and examples herein.

In certain examples, the method may include detecting generic E. coli, for instance from a food product, water, a combination thereof, and the like. Any of the water sample embodiments and examples herein may include potable drinking water, a waste water, an effluent, a surface water, a ground water, a swimming water, an irrigation water, a run off, a combination thereof, and the like. For instance, the method may include detecting greater than about one CFU/mL E. coli in about one hundred mL water. Further, the method may include detecting E. coli in water within less than about six hours. The method may include detecting greater than about one CFU/mL E. coli in about twenty-five grams of a food product. Additionally, the method may include targeting nucleic acid sequence specific to T type bacteriophages, for example T-4, and multiplexing the T type bacteriophages to detect bacteria such as generic E. coli . Those skilled in the art having the benefit of this disclosure will recognize additional enhanced detection levels and test result times supported by the embodiments and examples herein.

In certain examples, testing may be enhanced by selecting bacteriophage for a specificity to the bacterial presence to be analyzed. Further, the step of adding bacteriophage may be enhanced for a specific predetermined detection by adding a distinct bacteriophage with a specificity to a different bacteria within the same sample. Those of ordinary skill having the benefit of this disclosure will recognize additional analytes of interest.

In particular examples, the lateral flow test strip may include a solid backing support. The lateral flow test strip may include a nitrocellulose membrane adhered to the solid backing support. The lateral flow test strip may include an overlay having a transparent tape laminated onto the test strip and adapted to prevent contamination and drive sample flow along the test strip. The overlay may be aligned over the filtration conjugate pad to define an exposed filtration conjugate pad segment and a concealed filtration conjugate pad segment. The overlay may be aligned over substantially half of the filtration conjugate pad.

In some examples, the sample collection device and the lateral flow test strip may be a self-contained testing system having a visible line test result display. The sample collection device and the lateral flow test strip may provide rapid detection of bacteria, and the like, in dairy, including, but not limited to, raw milk samples.

In particular examples, the sampling probe may include a swab device having a handling portion and a sampling distal end. The vial may include an extraction buffer. The vial may be removable about the sample collection device. The sample collection device and the lateral flow test strip may provide rapid detection of bacteria, or the like, in dairy samples. The sample collection device and the lateral flow test strip may be housed in a single-use assembly. The sample collection device and the lateral flow test strip may be a self-contained testing system having at least one visible line test result display.

In particular examples, the control line may include a capture agent with affinity to the antibody label complex independent of the nucleic acid sequence label complex being bound by respective analytes. The control line may display a valid sample flow. The test strip may include a nucleic acid sequence/loop protein complex adapted to create a mobile phase admixture when contacted with the sample. The nucleic acid/protein complex may include a gold-labeled nucleic acid/protein complex.

In one example, the lateral flow test strip includes a solid backing support. The lateral flow test strip may include a nitrocellulose membrane adhered to the solid backing support. The lateral flow test strip may include an overlay having a transparent tape laminated onto the test strip and adapted to prevent contamination and drive sample flow along the test strip. The overlay aligned over the filtration conjugate pad may define an exposed filtration conjugate pad segment and a concealed filtration conjugate pad segment. The overlay may include align over substantially half of the filtration conjugate pad.

The lateral flow test strip may be housed in a blister package substantially enclosing the test strip. The blister package may include a backing bottom surface and an upper blister bubble. In some examples, the control line displays a valid sample flow. The nucleic acid/protein complex may include a gold-labeled complex binder protein.

Example elements may include a test strip having a solid backing support; a nitrocellulose membrane adhered to the solid backing support and including at least one control area and at least one test area; a filtration conjugate pad having a top side and a fibrous bottom side and aligned to the nitrocellulose membrane at a contact point, and wherein the bottom fibrous side includes a labeled receptor; and an overlay enclosing the nitrocellulose membrane and the contact point between the nitrocellulose membrane and filtration conjugate pad.

Example elements may include an overlay comprising a transparent tape laminated onto the test strip to prevent contamination and drive sample flow along the test strip. For instance, the overlay may be aligned over the filtration conjugate pad to generally define an exposed filtration conjugate pad segment and a concealed filtration conjugate pad segment. For example, the overlay may be aligned over substantially half of the filtration conjugate pad. Further, the overlay may pressurize at least a portion of the test strip to generate an even flow of sample about the test strip.

Example elements may include a conjugate pad or other element containing sensitivity regulators adapted to adjust, through upregulation, downregulation, or the like the sensitivity of the test strip to one or more detection targets for which the test strip may be overly or underly sensitive.

Example elements may include a contact point includes an overlap of filtration conjugate pad onto the nitrocellulose membrane. For instance, the contact point includes between about two millimeters to about three millimeters of overlap of filtration conjugate pad onto the nitrocellulose membrane.

Example elements may include a solid support comprises a transparent material for directly viewing a result without equipment. In particular examples, the filtration conjugate pad includes a fiberglass pad or the like. The nitrocellulose membrane may include a plurality of control lines. Similarly, the nitrocellulose membrane may include a plurality of test lines.

In some examples, the method includes comparing intensity of a detectable signal at each of the test line and the control line, wherein a greater intensity of the detectable signal in any one test line as compared to the control line indicates a negative result for a particular analyte and a greater intensity of the detectable signal in the control line compared to any one test line indicates a positive result for the particular analyte, including bacterial detection or the like. In certain embodiments, comparing intensity of the detectable signals includes directly observing the test strip without equipment. Further, the method may include adjusting test sensitivity by adding a mixture of receptors to the test strip.

Yet another embodiment of the disclosure includes an assembly for the analysis of a sample, the assembly may include any of the test strip embodiments and examples shown and described herein and the delivery device embodiments and examples shown and described herein. For instance, the test strip may have a nitrocellulose membrane, a filtration conjugate pad overlapping a portion of the nitrocellulose membrane and including a fibrous bottom side with a sprayed bead labeled receptor, and an overlay tape enclosing the nitrocellulose membrane and a portion of the filtration conjugate pad; and the delivery device may have an elongated body and a receiving distal cavity comprising a surface tension to retain a predetermined volume of the sample during operation.

In one embodiment, an assembly for detecting bacteria in a sample includes a compartmentalized container having a sample collector reservoir; a first storage compartment cooperatively-engaged with the reservoir, and housing assay components in a storage position and releasably introducing the assay components into the reservoir to define a first admixture; a second storage compartment in operative communication with the reservoir, and housing ribonucleic acid (RNA) or deoxyribonucleic acid (DNA) extraction reagents in a storage position and releasably introducing the extraction reagents into the first admixture to define a second admixture; a second container adapted to receive a portion of the second admixture and having: a bacteriophage adapted to target a bacterial enzyme operon, or a specific viral nucleic acid sequence, thereby logarithmically multiplying at least one target RNA or DNA; a loop-mediated isothermal amplification system; and a clustered regularly interspaced short palindromic repeat (CRISPR) treatment system adapted to initiate a CRISPR of the RNA or DNA bound loop complexes, and wherein the assembly adapted to detect the loop complexes for rapidly predicting a bacterial presence in the sample.

In certain examples, the first storage compartment includes a moisture and vapor impervious layer enclosing the assay components. The second storage compartment may include a moisture and vapor impervious layer enclosing the extraction reagents. The second storage compartment may be cooperatively-engaged with the reservoir, a combination thereof, and the like. The assay components include any of the culture media, culture broth, induction agents, additives, reagents, and the like shown, described, and incorporated by reference herein. The assay components may include a magnetic bead. The magnetic bead may include a lectin, an antibody, a combination thereof and the like. Further, the assay components may include a phage.

In alternative embodiments, the second storage compartment may comprises an acidification assembly.

In certain examples, the bacterial presence may include a coliform in a dairy product selected from the group consisting of a raw milk and a pasteurized dairy product. The assembly may detect less than about ten CFU/mL coliform in the pasteurized dairy product. The assembly may detect less than about one hundred CFU/mL coliform in the raw milk. The assembly may detect coliform in pasteurized milk within less than about four hours. The assembly may detect a coliform in raw milk in less than about one hour.

In particular examples, the assembly may include multiplexing the nucleic acid sequences and the operons. The assembly may increase activity of the lactose operon thereby increasing a DNA, RNA, and protein production. The assembly may target B-galatosidase production. The assembly may include a loop amplification specific to a RNA or DNA sequence in coliphage. The assembly may include a lateral flow with visible nano-particles to detect loop complexes. The assembly may include operating FITC and fluorescence to detect loop complexes. The assembly may include operating luminescence chemistry adapted to detect the loop complexes. The assembly may include a loop-mediated isothermal amplification includes using a LACz gene.

In certain examples, the bacterial presence includes E. coli in a sample selected from the group consisting of a food product and a water. The assembly may include a detection of greater than about one CFU/mL E. coli in about one hundred mL water, for instance a detection of E. coli in water within less than about six hours. The sample may include a potable drinking water, a waste water, an effluent, a surface water, a ground water, a swimming water, an irrigation water, a run off, a combination thereof, and the like. The assembly may include a detection greater than about one CFU/mL E. coli in about twenty-five grams of a food product. The assembly may include targeting B-glucuronidase (GUS) production. The assembly may include multiplexing the nucleic acid sequences and the operons such as GUS and LAC. The assembly may include targeting the GUS operon thereby increasing RNA and protein production. The assembly may include at least one T type bacteriophage. The assembly may include a specific RNA or DNA sequence of the T type bacteriophage.

In particular examples, the assembly may include a lateral flow with a visible nano-particles to detect the loop complexes. The assembly may include FITC and fluorescence adapted to detect the loop complexes. The assembly may include luminescence chemistry adapted to detect the loop complexes. The sample collector reservoir may include a filter.

In one embodiment, a method for rapidly predicting a bacterial presence in a sample includes collecting a sample in a compartmentalized container housing assay components in a first independent compartment, and ribonucleic acid (RNA) or deoxyribonucleic acid (DNA) extraction reagents in a second independent compartment; introducing the assay components from the independent compartment into a sample reservoir for infecting the sample; introducing the extraction agents from the independent compartment for extracting a targeted RNA or DNA; initiating a loop-mediated isothermal amplification of the sample; initiating a clustered regularly interspaced short palindromic repeat (CRISPR) treatment of the RNA or DNA bound loop complexes; and detecting the loop complexes.

In particular examples, the method may include introducing the assay components into a sample reservoir via screwing, including, but not limited to, opposing threads of a cap about the container to break any seal, enclosure, or the like. The method may include extending a plunger, including two or more plungers, through any seal, enclosure, or the like to expose the first independent compartment. Further, introducing the assay components into a sample reservoir may include removing a seal on the first independent compartment. The seal may include a tab or the like. The method may include dissolving any of the assay components shown and described herein, including with agitation or without agitation.

In particular examples, the method may include incubating the reservoir. The method may include incubating the reservoir between about thirty degrees Celsius to about fifty degrees Celsius. The method may include introducing a phage for targeting an area within the bacteria. The method may include removing a liquid concentrating the bacteria. The method may include introducing the extraction reagents via screwing opposing threads of a second independent cap about the container. The method may include introducing the extraction reagents via extending a plunger to perforate the second independent compartment. Further, the method may include introducing the extraction reagents into a sample reservoir via removing a seal on the second independent compartment.

In certain examples, the method may include detecting a bacterial enzyme within less than about twenty minutes. The method may include detecting an aerobic bacteria in a dairy product selected from the group consisting of a raw milk and a pasteurized dairy product. The method may include detecting and semi-quantitating between about less than 5000 CFU/mL to about greater than 300000 CFU/mL aerobic bacteria. The method may include detecting and semi-quantifying between about 5000, about 20000, about 100000, and about 300000 CFU/mL aerobic bacteria. The method may include targeting a DNA or RNA to at least one respiratory enzyme in respiration. The method may include targeting a DNA or RNA to at least one respiratory enzyme in a KREBS cycle electron transport chain. The method may include targeting adenosine triphosphate (ATP) kinase. The method may include targeting NADH, pyruvate kinase, succinic anhydride, dehyrogenases, RNA, a DNA or RNA to at least one respiratory enzyme in a KREBS cycle electron transport chain, a combination thereof and the like.

In certain examples, the method may include operating a lateral flow with a visible nano-particles to detect the loop complexes. The method may include operating a FITC and a fluorescence to detect the loop complexes. The method may include applying luminescence chemistry to detect the loop complexes.

In particular examples, infecting the sample with a bacteriophage includes targeting a bacterial enzyme operon, or a specific viral nucleic acid sequence, and thereby logarithmically multiplying the RNA or DNA.

In one embodiment, an assembly for detecting bacteria in a sample includes a compartmentalized container having a sample collector reservoir; a first storage compartment cooperatively-engaged with the reservoir, and housing assay components in a storage position and releasably introducing the assay components into the reservoir to define a first admixture; a second storage compartment in operative communication with the reservoir, and housing ribonucleic acid (RNA) or deoxyribonucleic acid (DNA) extraction reagents in a storage position and releasably introducing the extraction reagents into the first admixture to define a second admixture, and housing a clustered regularly interspaced short palindromic repeat (CRISPR) treatment system adapted to initiate a CRISPR of the RNA or DNA bound loop complexes, and wherein the assembly adapted to detect the loop complexes for rapidly predicting a bacterial presence in the sample.

In one embodiment, preparing a sample for bacterial analysis includes infecting the sample with a bacteriophage. Typically, an enzyme operon, a specific viral nucleic acid sequence, and the like are replicated/multiplied using PCR transcriptional enzymes to create targeted nucleic acid such as ribonucleic acid (RNA), deoxyribonucleic acid (DNA), and the like. The method includes initiating a loop-mediated isothermal amplification of the sample, and then initiating a clustered regularly interspaced short palindromic repeat (CRISPR) treatment of the RNA or DNA bound loop complexes. The method may then include detecting the loop complexes. Thus, the methodology shown and described herein may rapidly predict a bacterial presence in the sample. For instance, the method may prepare a sample for rapid detection of at least one specific nucleic acid sequence, when the sequence is present and/or amplified, thereby predicting bacterial presence.

In certain examples, the method may include detecting coliform in a dairy product, such as raw milk, pasteurized dairy products, a combination thereof, and the like. For instance, the inventions herein may detect less than about ten CFU/mL coliform in a pasteurized dairy product. And for instance, the inventions herein may detect less than about one hundred CFU/mL coliform in a raw milk. In certain examples, the inventions herein may detect coliform in pasteurized milk within less than about four hours, for instance less than one hour and detecting coliform in raw milk in less than about one hour and for instance within approximately ten minutes or less. Those skilled in the art having the benefit of this disclosure will recognize additional enhanced detection levels and test result times supported by the embodiments and examples herein.

The method may include increasing activity of the lactose operon, for example a LAC operon, such as the LACz operon and the like, for instance thereby increasing a DNA, RNA, protein, and the like, production. The method may include targeting B-galatosidase production.

In certain examples, the method may include detecting aerobic bacteria in a dairy product. For instance, the dairy product may be a raw milk, a pasteurized dairy product, and the like. The method may include detecting and semi-quantitating between about less than 5000 CFU/mL to about greater than 300000 CFU/mL aerobic bacteria, for instance about 5000, about 20000, about 100000, and about 300000 CFU/mL aerobic bacteria. Those skilled in the art having the benefit of this disclosure will recognize additional enhanced detection levels supported by the embodiments and examples herein.

In certain examples, the method may include targeting a DNA or RNA to at least one respiratory enzyme in respiration. Further, the method may include targeting a DNA or RNA to at least one respiratory enzyme in a KREBS cycle electron transport chain. The method may include targeting adenosine triphosphate (ATP) kinase, NADH, pyruvate kinase, succinic anhydride, dehyrogenases, a combination thereof, and the like. In particular examples, the method may include multiplexing nucleic acid sequences to multiple enzyme operons.

In particular examples, the method may include detecting messenger RNA (mRNA).

In certain examples, the method may include detecting generic E. coli, for instance from a food product, water, a combination thereof, and the like. Any of the water sample embodiments and examples herein may include potable drinking water, a waste water, an effluent, a surface water, a ground water, a swimming water, an irrigation water, a run off, a combination thereof, and the like. For instance, the method may include detecting greater than about one CFU/mL E. coli in about one hundred mL water. Further, the method may include detecting E. coli in water within less than about six hours. The method may include detecting greater than about one CFU/mL E. coli in about twenty-five grams of a food product. Those skilled in the art having the benefit of this disclosure will recognize additional enhanced detection levels and test result times supported by the embodiments and examples herein.

In particular examples, the method may include targeting B-glucuronidase (GUS) production. Further, the method may include multiplexing multiple nucleic acid sequences to multiple enzymes and their operons including multiplexing the enzymes targeting the one or more GUS operons and one or more LAC operons, for example the LACz operon, alone or together or with other operons. For example, targeting GUS and LACz operons thereby increasing RNA, such as mRNA production, protein production, and the like. Target operons, such as LACz and GUS, can be selected with consideration to detection interferences. For example, a LAC operon may interfere with detection of all coliforms while a GUS operon may interfere with detection of other bacteria. Additionally, the method may include targeting nucleic acid sequence specific to T type bacteriophages, for example T-4, and multiplexing the T type bacteriophages to detect bacteria such as generic E. coli .

Unexpectantly, Applicants have discovered any of the methods, systems, and assemblies herein may include detecting RNA or DNA without an extraction step to enhance testing procedures and decrease testing times. For instance, the method may include detecting the bacterial nucleic acid sequences and/or enzymes without a centrifugation step. Further, the method may include detecting the nucleic acid sequences and/or bacterial enzyme without a filtration step.

In certain examples, testing may be enhanced by selecting bacteriophage for a specificity to the bacterial presence to be analyzed. Further, the step of adding bacteriophage may be enhanced for a specific predetermined detection by adding a distinct bacteriophage with a specificity to a different bacteria within the same sample.

Applicants have further unexpectantly discovered the logarithmic increase of viral inserted RNA decreases bacterial culturing and/or detection time. Therefore, the logarithmic increase of RNA may eliminate or reduce the time for a step of culturing bacteria.

In one embodiment, analysis of a sample includes infecting the sample with a bacteriophage, and wherein a bacteriophage nucleic acid inserts and activates a bacterial enzyme operon, or a specific viral nucleic acid sequence, thereby logarithmically multiplying the target of at least one selected nucleic acid sequence. The analysis may include initiating a loop-mediated isothermal amplification of the sample. Further, the analysis may include initiating a CRISPR treatment of the RNA or DNA bound loop complexes. The analysis may include detecting the loop complexes. In addition, the analysis may include detecting a pathogen from a source selected from the group consisting of a food sample and an environmental surface.

In certain examples, the analysis may include detecting at least one CFU/mL salmonella, at least one CFU/mL hemorrhagic E. coli, at least one CFU/mL Listeria, a combination thereof, and the like. Those of ordinary skill having the benefit of this disclosure will recognize additional analytes of interest.

In particular examples, the analysis may include detecting at least one pathogen in a twenty-five gram sample of food. Further, the analysis may include detecting a pathogen in a food sample in less than about eight hours. The analysis may include multiplexing specific nucleic acid sequences, enzymes and their operons and phage specific nucleotide sequences. In addition, the analysis may include rapidly amplifying DNA, RNA, targets for detection. The analysis may include introducing at least one bacteriophage specific to the pathogen, including introducing a plurality of bacteriophages specific to the pathogen.

In certain examples, the analysis may include initiating a loop amplification specific to a RNA or DNA sequence in a bacteriophage. Further, the analysis may include initiating lateral flow with a visible nano-particles to detect the loop complexes. The analysis may include initiating FITC and a fluorescence to detect the loop complexes. The analysis may include applying luminescence chemistry, for instance to detect the loop complexes. Further, the analysis may include colorimetric detecting using a spectrophotometer. The analysis may include using detection systems such as Charm Sciences, Inc. luminometer detection systems including the Charm EPIC system. The analysis may also include using a 96 well detection system or the like. Those of ordinary skill in the art having the benefit of this disclosure will recognize additional luminometer, concentrator, and the like systems and operations.

In one embodiment, a method for preparing a sample for analysis includes initiating a loop-mediated isothermal amplification of the sample; initiating a CRISPR of RNA or DNA bound loop complexes; and detecting the loop complexes, and wherein the method adapted for preparing the sample for rapid detecting of at least one bacterial nucleic acid and/or enzyme, when present, as predicting a presence and/or a semi-quantitative level of bacteria in the sample.

In one embodiment, a method of analysis of a dairy sample includes initiating a loop-mediated isothermal amplification of the sample; infecting the sample with a bacteriophage, and wherein a lactose operon logarithmically multiplying targeted RNA; and detecting the RNA and predicting an aerobic bacteria count of the dairy sample.

Those skilled in the art having the benefit of this disclosure will recognize a variety of lateral flow test strip, and similar assay, elements and embodiments useful and compatible with any of the methods and systems shown and described herein. Further, those skilled in the art having the benefit of this disclosure will recognize a variety of swab test sample apparatus elements and embodiments useful and compatible with any of the methods and systems shown and described herein.

Those skilled in the art having the benefit of this disclosure will recognize useful elements and nomenclature for the preparation of samples and analysis from enzymes https://www.broadinstitute.org/files/publications/special/COVID-19%20detection%20(updated).pdf ; https://www.journalofdairyscience.org/article/S0022-0302(17)30662-8/pdf ; https://www.ncbi.nlm.nih.gov/pmc/articles/PMC5300967 ; https://link.springer.com/article/10.1007/s11274-019-2601-5, which are herein incorporated by reference in their entireties.

In particular examples, the lateral flow test strip may include a solid backing support. The lateral flow test strip may include a nitrocellulose membrane adhered to the solid backing support. The lateral flow test strip may include an overlay having a transparent tape laminated onto the test strip and adapted to prevent contamination and drive sample flow along the test strip. The overlay may be aligned over the filtration conjugate pad to define an exposed filtration conjugate pad segment and a concealed filtration conjugate pad segment. The overlay may be aligned over substantially half of the filtration conjugate pad.

In some examples, the sample collection device and the lateral flow test strip may be a self-contained testing system having a visible line test result display. The sample collection device and the lateral flow test strip may provide rapid detection of bacteria, and the like, in dairy, including, but not limited to, raw milk samples.

In particular examples, the sampling probe may include a swab device having a handling portion and a sampling distal end. The vial may include an extraction buffer. The vial may be removable about the sample collection device. The sample collection device and the lateral flow test strip may provide rapid detection of bacteria, or the like, in dairy samples. The sample collection device and the lateral flow test strip may be housed in a single-use assembly. The sample collection device and the lateral flow test strip may be a self-contained testing system having at least one visible line test result display.

In particular examples, the control line may include a capture agent with affinity to the antibody label complex independent of the nucleic acid sequence label complex being bound by respective analytes. The control line may display a valid sample flow. The test strip may include a nucleic acid sequence/loop protein complex adapted to create a mobile phase admixture when contacted with the sample. The nucleic acid/protein complex may include a gold-labeled nucleic acid/protein complex.

In one example, the lateral flow test strip includes a solid backing support. The lateral flow test strip may include a nitrocellulose membrane adhered to the solid backing support. The lateral flow test strip may include an overlay having a transparent tape laminated onto the test strip and adapted to prevent contamination and drive sample flow along the test strip. The overlay aligned over the filtration conjugate pad may define an exposed filtration conjugate pad segment and a concealed filtration conjugate pad segment. The overlay may include align over substantially half of the filtration conjugate pad.

The lateral flow test strip may be housed in a blister package substantially enclosing the test strip. The blister package may include a backing bottom surface and an upper blister bubble. In some examples, the control line displays a valid sample flow. The nucleic acid/protein complex may include a gold-labeled complex binder protein.

Example elements may include a test strip having a solid backing support; a nitrocellulose membrane adhered to the solid backing support and including at least one control area and at least one test area; a filtration conjugate pad having a top side and a fibrous bottom side and aligned to the nitrocellulose membrane at a contact point, and wherein the bottom fibrous side includes a labeled receptor; and an overlay enclosing the nitrocellulose membrane and the contact point between the nitrocellulose membrane and filtration conjugate pad.

Example elements may include an overlay comprising a transparent tape laminated onto the test strip to prevent contamination and drive sample flow along the test strip. For instance, the overlay may be aligned over the filtration conjugate pad to generally define an exposed filtration conjugate pad segment and a concealed filtration conjugate pad segment. For example, the overlay may be aligned over substantially half of the filtration conjugate pad. Further, the overlay may pressurize at least a portion of the test strip to generate an even flow of sample about the test strip.

Example elements may include a conjugate pad or other element containing sensitivity regulators adapted to adjust, through upregulation, downregulation, or the like the sensitivity of the test strip to one or more detection targets for which the test strip may be overly or underly sensitive.

Example elements may include a contact point includes an overlap of filtration conjugate pad onto the nitrocellulose membrane. For instance, the contact point includes between about two millimeters to about three millimeters of overlap of filtration conjugate pad onto the nitrocellulose membrane.

Example elements may include a solid support comprises a transparent material for directly viewing a result without equipment. In particular examples, the filtration conjugate pad includes a fiberglass pad or the like. The nitrocellulose membrane may include a plurality of control lines. Similarly, the nitrocellulose membrane may include a plurality of test lines.

In some examples, the method includes comparing intensity of a detectable signal at each of the test line and the control line, wherein a greater intensity of the detectable signal in any one test line as compared to the control line indicates a negative result for a particular analyte and a greater intensity of the detectable signal in the control line compared to any one test line indicates a positive result for the particular analyte, including bacterial detection or the like. In certain embodiments, comparing intensity of the detectable signals includes directly observing the test strip without equipment. Further, the method may include adjusting test sensitivity by adding a mixture of receptors to the test strip.

Yet another embodiment of the disclosure includes an assembly for the analysis of a sample, the assembly may include any of the test strip embodiments and examples shown and described herein and the delivery device embodiments and examples shown and described herein. For instance, the test strip may have a nitrocellulose membrane, a filtration conjugate pad overlapping a portion of the nitrocellulose membrane and including a fibrous bottom side with a sprayed bead labeled receptor, and an overlay tape enclosing the nitrocellulose membrane and a portion of the filtration conjugate pad; and the delivery device may have an elongated body and a receiving distal cavity comprising a surface tension to retain a predetermined volume of the sample during operation.

Any of the test strips herein may communicate, including, but not limited to, dipped, partially submerged, and in certain alternative examples fully submerged, or the like into vial, or similar sample delivery device, to enhance testing efficiency and minimize contamination.

The above summary was intended to summarize certain embodiments of the present disclosure. Embodiments will be set forth in more detail in the figures and description of embodiments below. It will be apparent, however, that the description of embodiments is not intended to limit the present inventions, the scope of which should be properly determined by the appended claims.

### BRIEF DESCRIPTION OF THE DRAWINGS

Embodiments of the disclosure will be better understood by a reading of the Description of Embodiments along with a review of the drawings, in which:

FIG. 1 is a side view of one embodiment according to the present disclosure;

FIG. 1a is a side perspective view of the embodiment introduced in FIG. 1, with elements removed for clarity;

FIG. 2 is a side, partially exploded view of one embodiment according to the present disclosure;

FIG. 2a is a side, partially exploded perspective view of the embodiment introduced in FIG. 2, with elements removed for clarity;

FIG. 3 is a top view of one embodiment according to the present disclosure;

FIG. 3a is a side perspective view of the embodiment introduced in FIG. 3;

FIG. 3b is a side view of the embodiment introduced in FIG. 3;

FIG. 4 is a bottom perspective view of one embodiment according to the present disclosure in an operating position;

FIG. 4a is a bottom perspective view of the embodiment introduced in FIG. 4 in an operating position;

FIG. 5 is a side view of one embodiment according to the present disclosure with elements removed for clarity;

FIG. 5a is a top perspective view of one embodiment of a device according to the present disclosure in an operating position;

FIG. 6 is a side view of one embodiment according to the present disclosure;

FIG. 6a is a top perspective view of the embodiment introduced in FIG. 6 ;

FIG. 6b is a side, partially exploded perspective view of the embodiment introduced in FIG. 6;

FIG. 6c is a side, partially exploded perspective view of the embodiment introduced in FIG. 6;

FIG. 7 is a top perspective view of one embodiment according to the present disclosure;

FIG. 8 is a top perspective view of one embodiment according to the present disclosure;

FIG. 8a is a top perspective view of the embodiment introduced in FIG. 8;

FIG. 8b is a top perspective view of the embodiment introduced in FIG. 8 in an operating position; and

FIG. 8c is a top perspective view of the embodiment introduced in FIG. 8 in an operating position.

### DESCRIPTION OF EMBODIMENTS

In the following description, like reference characters designate like or corresponding parts throughout the several views. Also in the following description, it is to be understood that such terms as “forward,” “rearward,” “left,” “right,” “upwardly,” “downwardly,” and the like are words of convenience and are not to be construed as limiting terms.

Referring now to the drawings in general, it will be understood that the illustrations are for the purpose of describing embodiments of the disclosure and are not intended to limit the disclosure or any invention thereto. As shown and described herein, various methods, systems, and types of testing apparatuses helpful to predict a bacterial presence may be used for any of the examples and embodiments herein. For instance, those of ordinary skill in the art having the benefit of this disclosure will recognize that a variety of testing instruments, devices, and systems may be useful with any of the inventions herein, including but not limited to, TEST DEVICE, METHOD AND ASSEMBLY (U.S. Application No. 15/ 434399); SAMPLING METHOD AND DEVICE (U.S. Pat. No. 7,993,871); METHOD FOR ADJUSTING ANTIBIOTIC SENSITIVITY TO A TEST CULTURE (U.S. Pat. 7,897,365); INHIBITION ASSAY METHOD AND DEVICE FOR DETECTION OF ANTIBIOTICS (U.S. Pat. No. 8,476,064); LATERAL FLOW ASSAY ANALYSIS (U.S. Application No. 13/ 819064); IMPROVED LUMINOMETER AND CHAMBER (U.S. Application No. 14/ 154516); DETECTION SENSOR SYSTEMS AND METHODS (U.S. Application No. 14/ 207896); DYNAMIC PLATE READER (U.S. Application No. 14/ 480994); LUMINOMETER AND CHAMBER (U.S. Application No. 14/ 662825); METHOD AND APPARATUS FOR REDUCING LUMINESCENT TEST RESULT INTERFERENCES (U.S. Pat. No. 8663975); RESEALABLE MOISTURE TIGHT CONTAINER (U.S. Pat. No. 9493288); PHOTOMETER FOR USE WITH A TEST SAMPLE HOLDER (US Design Patent No. D393,601), U.S. Pats.5,965,453; 5,827,675; 5,985,675; 6,180,395; 6,319,446; 6,475,805; 7,097,983; 7,410,808; 7,785,899; 7,785,899; 7,897,365; 8,481,334; 8,592,171; 8,592,171; 8,592,171; and 9,057,724, any of the useful testing instrument features and elements are incorporated herein by reference.

The compartmentalized assay development device enclosures/containers 10, 100, 200 shown and described herein may include any size, configuration, dimension, and arrangement. As illustrated in the various figures, the compartmentalized assay development devices may generally have a closed bottom, a top, a sidewall, an opening, wherein the bottom, top, and sidewall define an interior in combination with any of the closures, caps and compartments shown and described herein. Further, any of the devices herein may be a single-use, disposable container, bag, or the like.

Any of the storage compartments herein may be removably fitted within a cap, addition, enclosure, or the like in an operative communication with the device, including with the sample reservoir. The storage compartments may have moisture, vapor, and the like impervious layers, films, etc. to support any of the storage shown and described herein. Certain storage compartments may have a soluble seal, membrane, etc. to deliver contents into the container as shown and described herein.

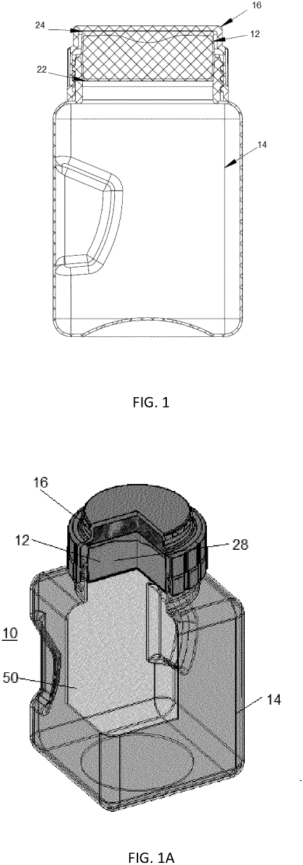

Various embodiments of compartmentalized assay development devices are shown and described herein. As illustrated in the FIGS. 1-5 and 6-7, various devices/assemblies may generally have a closed container bottom, a container top, a sidewall, a container opening, wherein the bottom, top, and sidewall define an interior in combination with any of the caps and compartments shown and described herein. Further, any of the containers herein may be a single-use, disposable substantially rigid container.

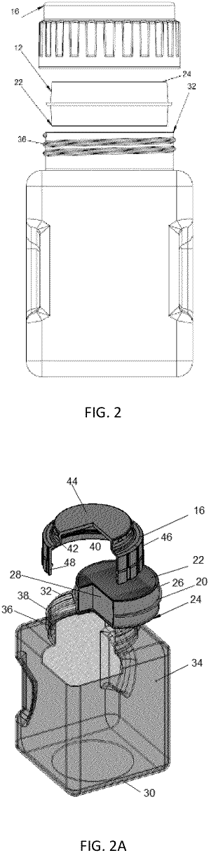

FIGS. 1-2a illustrate examples of one embodiment of a compartmentalized assay development device. As shown, the device may include a cap 16, a niblet 12, and a container 14. Certain embodiments of the cap may include an inner cavity 40, top surface 44, outer sidewall 46, inner periphery 42, and inner threads 48 to mate with the respective container threads. For instance, the container may include a neck 32 having outer threads 36, and an inner ledge 38. The container may include sidewalls 34 and a bottom 30.

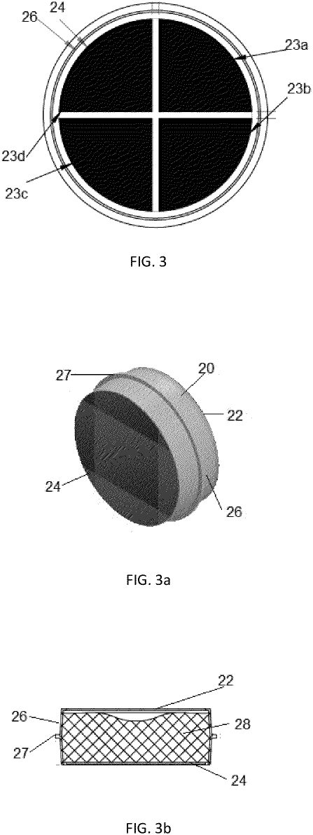

As shown, particular examples include a niblet 12, or aligned niblets, having an outer moisture impervious layer 22, for instance a foil seal or the like. The moisture impervious layer may include a sidewall 20. Particular examples include an alignment ridge 27 to mate with corresponding cap 16 or container 14 alignment positions. The storage receptacle 26 may house any of the assay and testing components shown and described herein. Applicant has unexpectedly discovered advantages of a niblet storage receptacle 26 having a multi component compartments, for instance 23a, 23b, 23c, 23d as illustrated in FIG. 3, to house and deliver any of the assay and testing components shown and described herein. As shown in FIG. 3a, the niblet 12 may include soluble film 24. Further shown in FIG. 3b, the niblet 12 may include a niblet wall 26, cavities 28 for substance in the niblet Those skilled in the art having the benefit of this disclosure will recognize additional multi component designs and arrangements to deliver any of the testing elements shown and described herein from a storage position to a testing operation position.

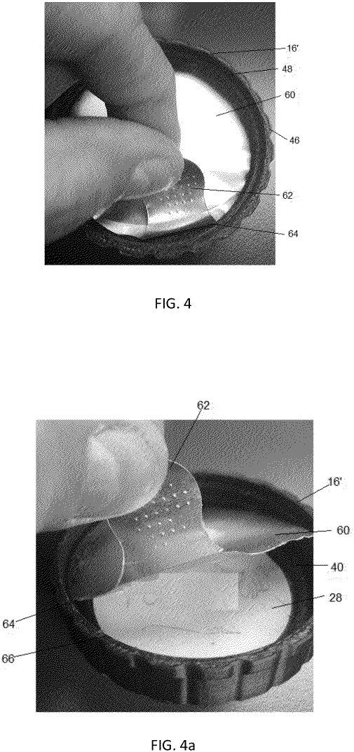

FIGS. 4a-4b introduce another embodiment of a compartmentalized assay development device. As shown, the peel cap embodiments may have a moisture impervious foil seal 60 removable from a periphery 48, for instance by pulling a pull tab 62 useful for any of the embodiments and examples shown and described herein. As illustrated, the device may include cap grip 46, foil tabs 64, contents storage cavity 28, disposable film 66, foil tab 64. Those skilled in the art having the benefit of this disclosure will recognize additional systems and collaboration with container and cap elements shown and described herein.

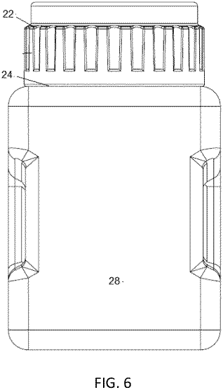

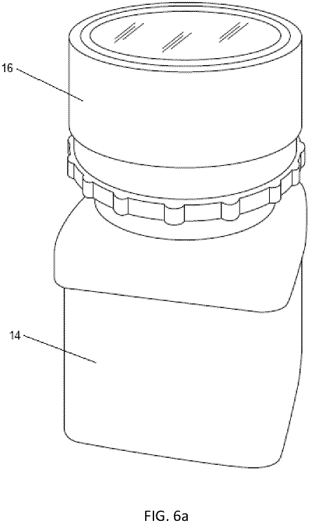

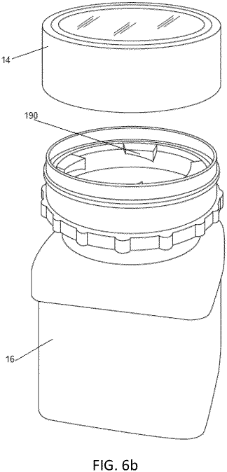

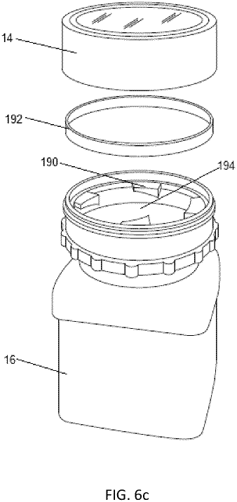

FIGS. 6-6c introduce another embodiment of a compartmentalized assay development device. As shown, the device includes a screwcap 24 on the cap 16 to manually deliver any of the assay and testing components shown and described herein. As illustrated, the cap 16 includes a removable impact ring 192 and cutting blades 194 to cut a seal/foil layer to deliver assay and testing contents into aperture 194 within container 16. Those skilled in the art having the benefit of this disclosure will recognize additional delivery devices and collaboration between container and cap elements shown and described herein.

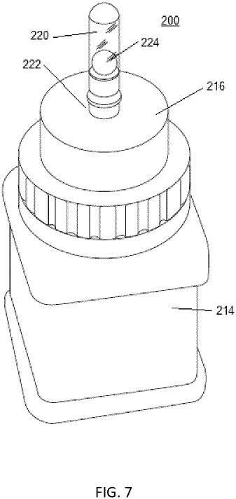

FIG. 7 introduces another embodiment of a compartmentalized assay development device 200. As shown, the device 200 includes a vial 220 protruding through an upper surface 222 of cap 216 above enclosure housing 214. The vial 220 may include at least one tablet 224, for instance any of the luminescent and/or assay and testing components shown and described herein. In certain examples, the vial 220 may collect a portion of a reaction admixture, for instance after a predetermined incubation, for any of the testing/analysis shown and described herein. Those skilled in the art having the benefit of this disclosure will recognize additional vial and device collaboration elements shown and described herein.

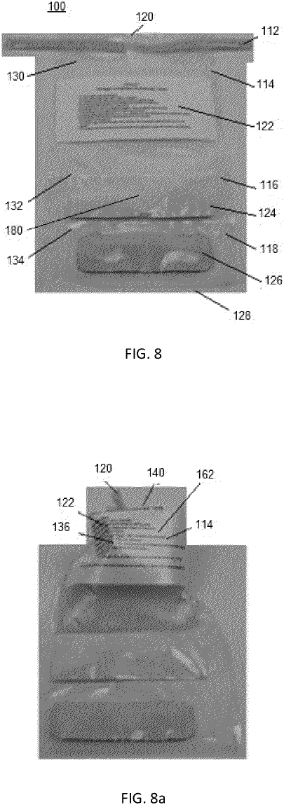

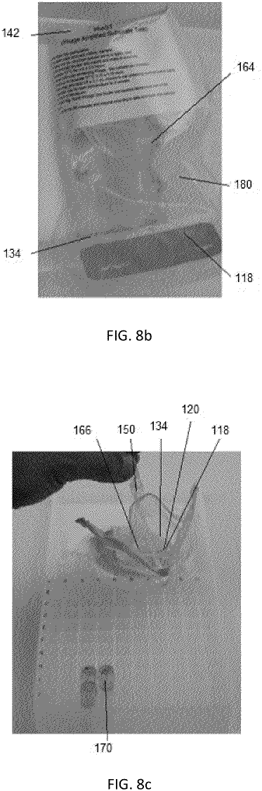

FIGS. 8-8c introduce another embodiment of a compartmentalized assay development device 100, including, but not limited to, a bag-style device. As shown in FIG. 8, assay development device 100 may have a proximate opening 112 to receive a sample and a variety of compartmentalized portions to provide the structure and assay elements for any of the analysis and testing shown and described herein. FIG. 8 illustrates one embodiment of assay development device 100 having a first compartment 114, a second compartment 116, and a third compartment 118; however, those skilled in the art having the benefit of this disclosure will recognize any variety of compartment configurations, dimensions, and arrangements, including greater or less than three compartments.

As shown in FIG. 8, the first compartment 114, for instance sample collector reservoir, is cooperatively-engaged with proximate opening 112 to receive a sample. The second compartment 116, for instance a media rehydrating compartment, is cooperatively-engaged with first compartment 114, for instance a sample collector reservoir. Further, third compartment 118, for instance a phage amplification compartment, is cooperatively-engaged with second compartment 116, for instance a media rehydrating compartment.

Any of the various compartments/reservoirs may be separated from one another with a temporary barrier, or the like, to provide a particular sequential analysis shown and described herein. As illustrated in FIGS. 8-8c, the proximate opening 112 may include a sanitary seal barrier 130, including, but not limited to, a perforation, recloseable seal, fastener 120, a combination thereof, or the like. A first temporary barrier 132 may be aligned between the first compartment 114, for instance sample collector reservoir, and the second compartment 116, for instance a media rehydrating compartment. The first temporary barrier 132 may be moisture impervious to compartmentalize, including separating, activity in the first compartment 114 with downstream activity in the second compartment 116. In particular examples, the first temporary barrier 132 may be moisture impervious burst liner, for instance a pressure sensitive liner, or the like, to facilitate cooperative engagement between the adjacent compartments. Similarly, second temporary barrier 134 may be aligned between the second compartment 116, for instance media rehydrating compartment, and the third compartment 118, for instance a phage amplification compartment. The second temporary barrier 134 may be moisture impervious to compartmentalize, including separating, activity in the second compartment 116 with downstream activity in the third compartment 118. In particular examples, the second temporary barrier 134 may be moisture impervious burst liner, or the like, to facilitate cooperative engagement between the adjacent compartments. Those skilled in the art having the benefit of this disclosure will recognize different barrier and compartment configurations, dimensions, and arrangements.

The various compartmentalized assay development devices prepare a sample for detection of bacteria, particular analytes, and the like, when present, in the sample. Therefore, compartments/reservoirs may house assay components to facilitate a testing sequence and minimize operator contact, contamination, and the like. As shown in FIGS. 8-8c, the first compartment 114, for instance sample collector reservoir, may house assay components 122, including, but not limited to, disinfectant neutralizers and the like. The second compartment 116, for instance media rehydrating compartment, may house media components 124, including, but not limited to, non-selective mediums for generic E. coli . In particular examples, the media 124 may be a Lauria-Bertani broth for salmonella testing and the like, while other media 124 examples may include a buffered peptone water broth for ground beef testing and the like. In certain examples, as shown in FIG. 8a, the second compartment 116 may house any of the phage amplification enclosures 180 shown and described herein. The third compartment 118, for instance a phage amplification compartment, may house amplification components 126, including, but not limited to, bactericide, buffers, and the like to facilitate testing and/or indicate biologics. And as shown in FIG. 8, the third compartment 118 may house any of the phage amplification enclosures 180 shown and described herein.

In certain embodiments shown and described herein, the assemblies and devices prepare a sample for detection of bacteria, or the like, when present, in a sample. For instance, the prepared sample may detect a recombinant luciferase enzyme. Those skilled in the art having the benefit of this disclosure will recognize the detection of other enzymes and the like. The recombinant luciferase enzyme may be an insert into an infecting phage, and thus may be replicated as part of a virus replication. In certain examples, the phage amplification may target at least one specific bacterial receptor, insert into an enzyme operon, replicate at least one specific nucleic acid sequence. In certain examples, a luciferase assay substrate may assist the detectable luminescent signal in the prepared sample as shown and described herein. In certain examples, the phage amplification includes a nanoluciferase, for instance a nanoluciferase having an indicator gene and a bacteriophage late promoter to control transcription of the indicator gene, such as described in METHODS AND SYSTEMS FOR RAPID DETECTION OF MICROORGANISMS USING INFECTIOUS AGENTS U.S. Pat.No. 10,519,483); IMIDAZO[1,2-α]PYRAZINE DERIVATES (U.S. Pat. No. 8,809,529); and https://www.promega.com/products/luciferase-assays/reporter-assays/nano_glo-luciferase-assaysystem and Nano-Glo® Luciferase Assay System Technical Manual Instructions for Use of Products N1110, N1120, N1130 and N1150 (NANO-GLO® is Registered trademark of Promega Corporation of Wisconsin), which are incorporated by reference herein in their entireties.

In certain examples, the phage amplification enclosure 180 may target a bacterial enzyme operon, or a specific viral nucleic acid sequence, to produce any of the amplifications shown and described herein. The phage amplification enclosure 180 may include a puncturable, including squeezable or the like, packet, vial, or similar enclosure that is generally adapted to release in the designated compartment. The phage amplification enclosure 180 may include at least one buffer.

Any of the various sample preparation and testing sequences may include incubation. Therefore, the compartmentalized assay development devices may provide structural integrity and material to withstand incubation and the like. In certain examples, a procedure includes incubating the sequence during activity in the second compartment 116. Further, a procedure includes incubating the sequence during activity in the third compartment 118. While other examples include incubation at particular sequences and during particular activities in certain compartments/reservoirs.

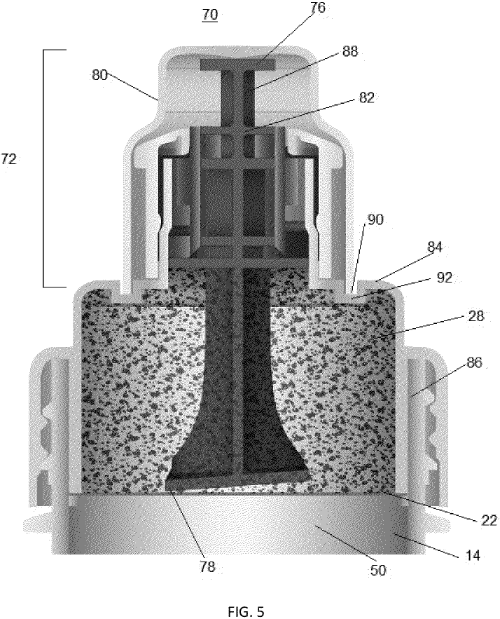

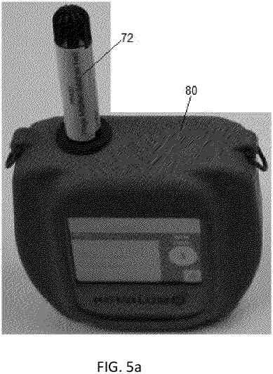

A luminometer 80 (shown in FIG. 5a), or similar device, may analyze any of the test sample preparations produced from the various compartmentalized assay development devices herein. For instance, at least a portion of the test sample preparations, including a phage amplified admixture 166, may be removed, including, but not limited to, pipetted 150, into a vial 170, or the like, and the luminescent substrate analyzed. The luminometer may analyze the luminescent substrate in the vial for rapidly predicting a bacterial presence, when present, in the sample. The luminescent signal may enable a semi-quantitative test result. The luminescent signal may be detectable by any handheld, benchtop, combination, or the like luminometer. Further, in certain examples, the vial may be delivered into the luminometer 80 via a delivery device 72, or the like. Those skilled in the art having the benefit of this disclosure will recognize different testing/analysis devices, tools, and procedures.

Numerous characteristics and advantages have been set forth in the foregoing description, together with details of structure and function. Many of the novel features are pointed out in the appended claims. The disclosure, however, is illustrative only, and changes may be made in detail, especially in matters of shape, size, and arrangement of parts, within the principle of the disclosure, to the full extent indicated by the broad general meaning of the terms in which the general claims are expressed. It is further noted that, as used in this application, the singular forms “a,” “an,” and “the” include plural referents unless expressly and unequivocally limited to one referent.

We claim what is claimed is:

1. A compartmentalized assay development device comprising:
a. a sample collector reservoir cooperatively-engaged with an opening and adapted to receive a sample;
b. a media rehydrating compartment cooperatively-engaged in communication capability with said sample collector reservoir and housing a media; and
c. a phage amplification compartment cooperatively-engaged in communication capability with said media rehydrating compartment, and
wherein said compartmentalized assay development device adapted to expose a phage amplification enclosure and prepare said sample for detection of bacteria, when present, in said sample.

2. The device of claim 1, wherein
a. said sample collector reservoir housing assay components in a storage position and adapted to releasably introduce said assay components in an admixture;
b. said media rehydrating compartment housing assay components in a storage position; and
c. said phage amplification compartment housing assay components in a storage position and adapted to releasably introduce said assay components in an admixture.

3. The device of claim 1, wherein
a. said sample collector reservoir housing assay components in a storage position and adapted to releasably introduce said assay components in an admixture; and
b. said phage amplification compartment housing recombinant phage assay components in a storage position and adapted to releasably introduce said assay components in an admixture.

4. The device of claim 1, including a first temporary barrier between said sample collector reservoir and said media rehydrating compartment.

5. The device of claim 1, including a second temporary barrier between said media rehydrating compartment and said phage amplification compartment.

6. The device of claim 1, including a phage amplification enclosure housed in said media rehydrating compartment.

7. The device of claim 1, including a phage amplification enclosure housed in said phage amplification compartment.

8. The device of claim 1, including a recloseable proximate opening.

9. The device of claim 1, wherein said assay components include a disinfectant neutralizer.

10. The device of claim 1, wherein said prepared sample for detection of bacteria being adapted for detection of a recombinant luciferase enzyme.

11. The device of claim 10, wherein said recombinant luciferase enzyme being an insert into an infecting phage and being replicated as part of a virus replication.

12. The device of claim 1, wherein said phage amplification enclosure adapted to target at least one specific bacterial receptor, insert into an enzyme operon, replicate at least one specific nucleic acid sequence.

13. The device of claim 1, including a luciferase assay substrate and wherein said prepared sample adapted to detect a luminescent signal.

14. The device of claim 1, including at least one burst liner.

15. The device of claim 12, wherein said phage amplification enclosure includes a nanoluciferase.

16. The device of claim 15, wherein said nanoluciferase comprises an indicator gene and a bacteriophage late promoter adapted to control transcription of said indicator gene.

17. An assembly for detecting bacteria, when present, in a sample, said assembly comprising:
a. a sample collector compartment cooperatively-engaged with a proximate opening and adapted to receive a sample;
b. a media compartment cooperatively-engaged with said sample collector compartment and housing a media in a storage position, wherein said sample collector compartment adapted to releasably introduce said sample into said media compartment to define a first admixture;
c. an amplification compartment cooperatively-engaged with said media compartment, wherein said media compartment adapted to releasably introduce said first admixture into said amplification compartment and support phage amplification to define a luminescent enzyme; and
d. a disinfectant compartment cooperatively-engaged with said amplification compartment and adapted to releasably introduce a disinfectant.

18. The assembly of claim 17, wherein said phage amplification adapted to target at least one specific bacterial receptor, insert into an enzyme operon, replicate at least one specific nucleic acid sequence.

19. The assembly of claim 17, including a concentrator adapted to concentrate said phages.

20. An assembly for detecting bacteria in a sample, said assembly comprising:
a. a compartmentalized container having
i. a sample collector reservoir;
ii. a first storage compartment cooperatively-engaged with said sample collector reservoir, and housing assay components in a storage position and releasably introducing said assay components into said sample collector reservoir to define a first admixture;
iii. an second compartment cooperatively-engaged with said first storage compartment, wherein said first storage compartment adapted to releasably introduce said first admixture into said second compartment and support a phage amplification to define specific nucleic acid sequences; and
iv. a third storage compartment in operative communication with said second storage compartment, and housing ribonucleic acid (RNA) or deoxyribonucleic acid (DNA) extraction reagents in a storage position and releasably introducing said extraction reagents into said first admixture to define a third admixture, and
wherein said assembly adapted to prepare said sample for rapidly predicting a bacterial presence, when present, in said sample.
